(19) United States
(12) Patent Application Publication
(10) Pub. No.: US 2026/0026446 A1
(43) Pub. Date: Jan. 29, 2026

(54) METHOD FOR BREEDING RECESSIVE GENETIC MALE STERILE LINE OF SPOROPHYTE

(71) Applicant: Yunnan University, Kunming, Yunnan (CN)

(72) Inventors: Yi ZHANG, Kunming, Yunnan (CN); Yun CHEN, Kunming, Yunnan (CN); Shuanglin DU, Kunming, Yunnan (CN)

(21) Appl. No.: 18/043,205

(22) PCT Filed: Aug. 26, 2021
(86) PCT No.: PCT/CN2021/114635

§ 371 Date: Feb. 27, 2023

(30) Foreign Application Priority Data
Aug. 27, 2020 (CN) 202010879352.4

(51) Int. Cl. A01H 1/00; A01H 1/02

(52) U.S. Cl. CPC A01H 1/12; A01H 1/023; A01H 1/123

(57) ABSTRACT

The present disclosure discloses a method for breeding a recessive genetic male sterile line of a sporophyte. The method comprises the following steps: linking a sporophyte male fertile gene Y, a down-regulation expression element Xi of an endogenous gametophyte male fertile gene, a herbicide A resistance gene A^{R}, a down-regulation element Bi of a herbicide B resistance gene, and an anthocyanin gene C or a down-regulation element Ci thereof, and transferring the linked genes into a sterile mutant of the sporophyte fertile gene Y, and pollinating a sterile plant with a positive plant to breed a genetic sterile line of a sporophyte. The method is universal and suitable for all plants, can obtain a genetic sterile line of a sporophyte with a sterile plant rate of 100%, reduce a cost for separating a sterile line from a maintainer line, identify purity intuitively, rapidly, accurately, and economically at an early stage.

### TECHNICAL FIELD

The present disclosure belongs to the field of genetic engineering and relates to a method for breeding a genetic male sterile line of a sporophyte. The method can breed a genetic sterile line of a sporophyte with a sterile plant rate of 100%, is conductive for using genetic sterility of a sporophyte in production of a hybrid seed, and fully utilizes a heterosis.

### REFERENCE TO AN ELECTRONIC SEQUENCE LISTING

The contents of the electronic sequence listing (SequenceListing.txt; Size: 48,199 bytes; and Date of Creation: Jul. 25, 2025) is herein incorporated by reference.

### BACKGROUND

Heterosis is a phenomenon where a first generation of a hybrid is superior to parents in yield, quality, and/or resistance, and is widely present in the biological world. The hybrid is a first generation generated by hybridization of two different parents and is a variety bred by utilizing heterosis. Hybrid seed production requires outcross pollination of both parents. However, a conventional seed production corresponding thereto does not require outcross pollination. In paddy rice, corn, rape, and other crops, use of the heterosis is an important strategy for achieving high yield, high resistance, and high quality. A male sterile line is a main way of utilizing heterosis. Recessive male genetic sterility insensitive to environments such as temperature and light is an important sterility type, and has the following advantages compared with cytoplasmic-nuclear interaction sterility and temperature-light sensitive genetic sterility: matching is free, all fertile materials can be matched with the sterile line to generate a hybrid with a normal fertility, such that heterosis can be fully utilized; and the fertility is stable and not influenced by natural environments such as temperature and light, and seed production is free of risks. However, in a natural state, the recessive genetic sterility insensitive to temperature and light cannot produce a sterile population with a sterile plant rate of 100%, and therefore is difficult to be applied to production of a hybrid. Pioneer company links a nucleotide sequence of a targeting sequence targeting an amyloid in corn to a front end of an amylase gene of corn, places the obtained nucleotide sequence under a pollen specific promoter PG47 to inactivate a pollen containing the component, links an exogenous fluorescent protein gene to facilitate identifying and sorting sterile and fertile seeds by a machine, and then links a sporophyte fertile gene to restore fertility of a sterile mutant of a related sporophyte. Therefore, the method is used for breeding a genetic sterile line of a sporophyte in paddy rice, corn and other crops (Chang et al., 2016; Wu et al., 2016). In order to reduce use of an exogenous gene, reduce a cost of distinguishing a sterile plant from a fertile plant, improve purity of a sterile line, and increase a method for breeding a sterile line of a sporophyte, more and new elements and methods for breeding a sterile line of a sporophyte are required to be designed and identified, and use of male genetic sterility of a sporophyte in hybrid breeding is accelerated and expanded. This is very important for fully utilizing heterosis and improving yield, quality, resistance and the like of grains.

### SUMMARY

In view of the above, the present disclosure aims to provide a novel method for breeding a sterile line of a sporophyte, and identifying and improving purity of the bred sterile line of a sporophyte.

In order to achieve the aforementioned objective, the present disclosure provides the following technical solutions.
1. A method for breeding a recessive genetic male sterile line of a sporophyte, comprising the following steps: linking a sporophyte male fertile gene Y, a down-regulation expression element Xi of an endogenous gametophyte male fertile gene, a herbicide A resistance gene A^{R}, a down-regulation element Bi of a herbicide B resistance gene, an anthocyanin gene C or a down-regulation element Ci thereof, and transferring the linked genes into a sterile mutant of the sporophyte fertile gene Y, and pollinating a sterile plant with a positive plant to breed a genetic sterile line of a sporophyte, and
specifically comprising the following steps:
(1) transferring an expression cassette containing a herbicide A resistance gene A^{R}, a down-regulation element Bi of a herbicide B resistance gene, a sporophyte male fertile gene Y capable of restoring fertility of a sporophyte sterile mutant, a down-regulation expression element Xi of an endogenous gametophyte male fertile gene, and an element C/Ci controlling synthesis of anthocyanin at the same time into a sterile mutant of a sporophyte male fertile gene Y to obtain a male fertile transgenic plant;
(2) selfing the male fertile transgenic plant, respectively spraying a herbicide A and a herbicide B, wherein a plant resisting the herbicide A as a male parent (maintainer line) and a plant resisting the herbicide B as a female parent (sterile line), and harvesting hybrid seeds, that is a bred genetic sterile line of a sporophyte; and the maintainer line and the sterile line can also be selected by color and are subjected to hybridization to breed a sterile line.

The sporophyte sterile mutant Y can be generated spontaneously or through mutagenesis. The mutagenesis comprises physical, chemical, and genetic engineering means such as rays, EMS and gene editing. Preferably, in the present disclosure, the sporophyte sterile mutant Y is a sporophyte sterile material Zhongjiu B-osabcg15 generated by backcross using a male sterile mutant naturally generated by an OsABCG15 gene as a sterile donor and Zhongjiu B as a recurrent parent; the sporophyte male fertile gene is an essential gene, the OsABCG15 gene, for pollen development of paddy rice; and the endogenous gametophyte male sterile interference sequence is an interference sequence of a gametophyte male sterility-related gene OsPTD1 gene.

The OsPTD1 (Loc_Os05g40740) gene (a genome sequence from an initiation codon to a termination codon is shown in SEQ ID NO. 1), a down-regulation solution is RNAi, a promoter of the RNAi is a pollen expression promoter, preferably a pollen specific promoter, more preferably a self promoter. The example of the present disclosure selects an OsPTD1 self promoter (a sequence is shown in SEQ ID NO. 2), an interference target forward sequence is shown in SEQ ID NO. 3, a stem-loop sequence is a first intron of OsMYB76 (a sequence is shown in SEQ ID NO. 4), and a terminator is Tnos (a sequence is shown in SEQ ID NO. 5). The herbicide A resistance gene can be any one herbicide resistance gene of a Bar gene (Biolaphos Resistance) resistant to Basta (glufosinate ammonium), an OsTubA2 gene resistant to trifluralin, etc. The example of the present disclosure selects the Bar gene (a sequence is shown in SEQ ID NO. 6). In the example of the present disclosure, the sporophyte male fertile gene is selected from the OsABCG15 gene (a genome sequence is shown in SEQ ID NO. 7). An interference sequence of the herbicide B resistance gene is sensitive to a certain herbicide B. The example of the present disclosure selects bentazone as a herbicide and a sensitive element is an interference target (a sequence is shown in SEQ ID NO. 9) of an endogenous Bel gene (a sequence is shown in SEQ ID NO. 8), and an interfered promoter is a Bel self promoter (a sequence is shown in SEQ ID NO. 10). An arrangement sequence of the above-mentioned elements is preferably an alternate arrangement of the elements expressed in different parts and periods, so as to avoid influences of expressions of the elements expressed at the same time and in the same parts.

In the present disclosure, a nucleotide sequence of the Bar gene is shown in SEQ ID NO. 6; a nucleotide sequence of the OsABCG15 gene is shown in SEQ ID NO. 17; the interference sequence of the endogenous herbicide B resistance gene is shown in SEQ ID NO. 18; and the interference sequence of the OsPTD1 gene is shown in SEQ ID NO. 20.

In the present disclosure, the anthocyanin gene can be any anthocyanin gene capable of expressing a color or a down-regulation element thereof. A modified gene 20 OsMYB76R (a sequence is shown in SEQ ID NO. 11) of the anthocyanin essential gene OsMYB76 is selected in the example of the present disclosure.

The present disclosure further comprises identifying and purifying purity of a sterile line and specifically comprising the following steps: investigating a ratio of a colorless plant, that is an identification result of purity of a sterile line, by using a pigment character. With regard to a non-conformity sterile line, bentazone is used to remove an off-type plant to improve purity of a sterile line in a seedling stage (seedling bed stage).

The present disclosure further comprises breeding a maintainer line and specifically comprises collecting a hybrid male parent seed and spraying a herbicide A to a progeny, and a plant survived is the maintainer line.

A principle of breeding a sterile line and controlling purity is as follows: Zhongjiu B-osabcg15 shows no anthocyanin color in all organs due to a mutation of the gene OsMYB76 and shows purple in organs such as coleoptiles, leaf sheaths, stigmas and the like after a normal OsMYB76R gene is transferred. According to this, a transgenic positive plant can be determined. Since the gametophyte male sterile element and the sporophyte male fertile gene are transferred in a linkage way, all colored plants generate pollens, wherein half of the pollens have normal functions, but half of the pollens have no pollination capability. If the colorless mutant is pollinated by the colored plant, only the part of the pollens without a transgenic component can produce a progeny which theoretically shows sporophyte sterility due to a lack of the normal OsABCG15 gene. Due to a linkage transfer of the sensitive element of the herbicide B (such as the interference element Bi of the Bel gene), a fertile plant with a transgenic component can be killed by spraying the herbicide B (such as bentazone) on a transgenic progeny, a survived plant shows sporophyte sterility due to a lack of the normal OsABCG15 gene, such that a sterile line of a sporophyte is obtained. Due to a linkage transfer of the herbicide A resistance gene (such as the Bar gene), a sterile plant without a transgenic component can be killed by spraying the herbicide A (such as Basta) on a transgenic progeny, and a survived plant is a fertile plant, so as to obtain a sporophyte sterile maintainer line. The maintainer line screened by the herbicide A is used to pollinate the sterile plant screened by the herbicide B, so as to generate a large amount of a sporophyte sterile line with a sterile plant rate of 100% and a sporophyte sterile line is bred. Due to mechanical mixing or incomplete gametophyte sterility, the bred sporophyte sterile line may contain a maintainer line, and thus purity of the sterile line is reduced. At this time, the purity of the sterile line can be identified according to a proportion of plants without an anthocyanin color in the sterile line, and an off-type plant is killed by spraying the herbicide B, so as to finally obtain a sporophyte sterile line with a sterile plant rate of 100%.

A principle of breeding a maintainer line: since the gametophyte male fertile interference sequence is a regulatory element based on a pollen specific expression gene, all female gametes of a transgenic progeny can be normally fertile (transgenic and non-transgenic female gametes respectively account for half), and the transgenic progeny can be fertilized and fruited. However, only half of pollens of male gametophytes have pollination capability (pollens without a transgene). Besides, since the gametophyte male sterile element is linked with the herbicide A resistance gene, half of plants of the selfed progeny have a transgenic component, and simultaneously shows color, sporophyte male fertility, and gametophyte male sterility. Therefore, a maintainer line can be screened by color or spraying the herbicide A.

As a general technical solution, the method of the present disclosure is universal and can be used in various plants such as paddy rice, corn, rape and the like. The method comprises breeding a sterile line of a sporophyte and identifying and improving purity of the sterile line of a sporophyte. Main elements of a related vector are sporophyte fertile and gametophyte sterile elements, and other elements are auxiliary synergistic action elements, and an objective aim of breeding a sterile line of a sporophyte can be fulfilled when these elements are deleted. Therefore, reducing these elements is not departing from the scope defined by the claims of the present disclosure. The main and auxiliary regulatory elements involved in the method can be flexibly selected from elements with the same functions. A method for down-regulating a gene expression can be interference, antisense RNA, miRNA and the like. In order to improve a down-regulation efficiency, redundant genes and genes with the same functions can be down-regulated at the same time, an expressed target can be down-regulated, and the copy number is variable. The sequence and relative positions of loading of various fragments can be adjusted according to a specific situation. In addition, the name of the vector used in a statement of the method is used only for convenience of the statement. It is not limited to use other symbols to represent the relevant vector in practical use. Therefore, practical use of the above alternative content does not suggest substantial differences from the present disclosure, and therefore does not affect the protection of the present disclosure.

The present disclosure has the beneficial effects: the method is universal and can be used in various plants such as paddy rice, corn, rape and the like to breed a sporophyte genetic sterile line with a sterile plant rate of 100%, can rapidly, accurately, and economically screen a sterile line and a maintainer line at an early stage by spraying different herbicides, can utilize a pigment character to perform a purity identification of a sterile line intuitively, rapidly, accurately, and economically at an early stage, can rapidly and economically kill an off-type plant by spraying a herbicide in a seedling stage (seedling bed stage) for a sterile line with an off-type plant exceeding a standard, and thus enable a sterile plant rate to reach 100% and reduce loss. The involved nucleic acid fragments may all be endogenous to reduce a concern on transgene.

### BRIEF DESCRIPTION OF DRAWINGS

In order to make the objectives, technical solutions, and beneficial effects of the present disclosure clearer, the present disclosure provides the following drawings:

FIG. 1 is a map of a multiple cloning site modification of a vector; and

FIG. 2 is a schematic diagram of a method for breeding a genetic male sterile line of a sporophyte.

### DESCRIPTION OF EMBODIMENTS

The technical means used in the examples are conventional means well known to a person skilled in the art. The experimental methods in the following examples which are not specified with specific conditions are generally performed under conventional conditions, for example, conditions disclosed in Molecular Cloning Experiment Guide (4th Edition, published by the Cold Spring Harbor Laboratory) or Elaborately Compiled Molecular Biology Experiment Guide (5th Edition, Science Press) or conditions recommended by manufacturers. A full-length sequence of a related nucleotide or a fragment thereof can be obtained by PCR amplification, recombination or artificial synthesis.

The present disclosure will be further illustrated in connection with drawings and specific examples, such that a person skilled in the art can better understand and implement the present disclosure, but the listed examples are not taken as limitation of the present disclosure.

### Example 1 Construction of Vector

1) Loading of OsMYB76R Gene into pCAMBIA1301

According to a previous research, an OsMYB76 gene is an essential gene for synthesizing anthocyanin of paddy rice. In materials such as Zhongjiu B and other materials in paddy rice, due to a functional deletion mutation of the OsMYB76 gene, all organs do not show an anthocyanin color (Zhang Yi, 2009). An OsMYB76 gene was optimized, commonly used enzyme cutting sites were removed, an OsMYB76R gene was artificially synthesized (SEQ ID NO. 11), the OsMYB76R was used as a template and primers JCF1 (SEQ ID NO. 12)/JCR2 (SEQ ID NO. 13) were used for amplifying a target fragment to be recycled for later use, NcoI and BstEII were used for thoroughly cutting a GUS gene in pCAMBIA1301, and a skeleton was recycled for later use; and the OsMYB76R was loaded into the pCAMBIA1301 to obtain a vector 1301-JC through a homologous recombination method.

### 2) 1301-JC Multiple Cloning Site Modification

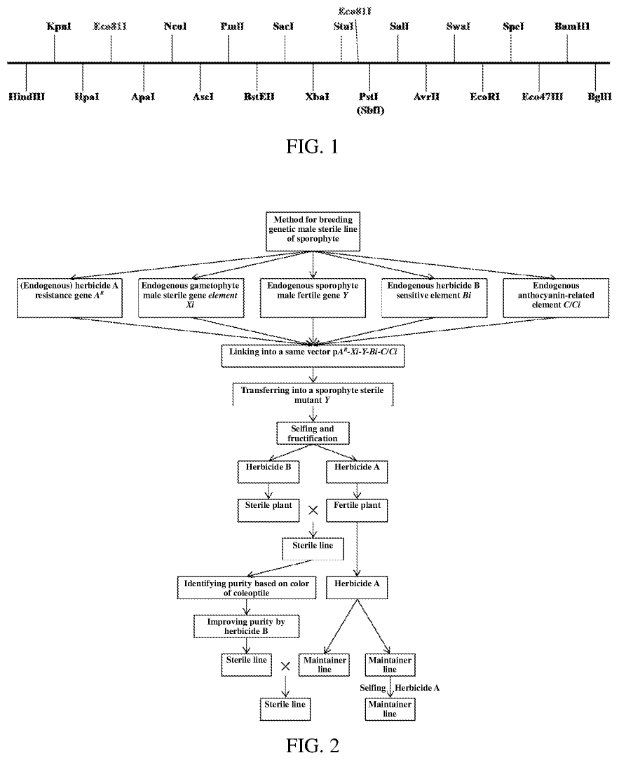

In order to conveniently load various regulatory elements, enzyme cutting sites and sequences of multiple cloning sites were redesigned (FIG. 1), a new multiple cloning site Y9755Gn (SEQ ID NO. 14) was artificially synthesized, the Y9755Gn was used as a template and primers RSF1 (SEQ ID NO. 15)/RSR1 (SEQ ID NO. 16) were used for amplifying a target fragment to be recycled for later use, EcoRI+HindIII were used for enzyme-cutting 1301-JC, and a skeleton was recycled for later use; and the target fragment was recombined into the skeleton to obtain a vector RSVMJC.

3) Loading of Bar Gene into RSVMJC

A Basta resistance gene Bar (SEQ ID NO. 6) was artificially synthesized, a 953 bp target fragment was cut from the Bar by using BstXI+XhoI to be recycled for later use, and the BstXI+XhoI were used for enzyme-cutting the RSVMJC at the same time, and a skeleton was recycled for later use; and the target fragment and the skeleton were linked to obtain a vector RSVMA^{R}JC.

4) Loading of OsABCG15 into RSVMA^{R}JC

A research shows that an OsABCG15 gene is an essential gene for pollen development of paddy rice. A mutant shows male genetic sterility of a pollen-free sporophyte (Wu et al, 2014). An OsABCG15 gene (SEQ ID NO. 7) was optimized, a part of introns and commonly used enzyme cutting sites were removed, a modified sequence TDS (SEQ ID NO. 17) was obtained by an artificial synthesis, SacI+SalI were used for cutting a target fragment 5,853 bp from the TDS to be recycled for later use, and the SacI+SalI were used for enzyme-cutting the RSVMA^{R}JC at the same time, and a skeleton was recycled for later use; and the target fragment and the skeleton were linked to obtain a vector pA^{R}-Y-C.

5) Loading of Bel Interference Element into pA^{R}-Y-C

A Bel gene (SEQ ID NO. 8) of paddy rice has an obvious resistance to bentazone and the paddy rice is easy to be killed by the bentazone after the Bel gene is mutated (Pan et al., 2006). An interference element of a bentazone resistance gene Bel was artificially synthesized at one time: a Bel self promoter (a sequence was shown in SEQ ID NO. 10)+a Bel gene interference target (a sequence was shown in SEQ ID NO. 9)+Tnos (a sequence was shown in SEQ ID NO. 5). A complete sequence Bi was shown in SEQ ID NO.18, SpeI+BamHI (incomplete enzyme-cutting) were used for cutting a target fragment 3,023 bp from Bi to be recycled for later use, the SpeI+BamHI were used for enzyme-cutting the pA^{R}-Y-C at the same time, and a skeleton was recycled for later use; and the target fragment and the skeleton were linked to obtain a vector pA^{R}-Y-Bi-C.

6) Loading of OsPTD1 Interference Element into pA^{R}-Y-Bi-C

A current research shows that an OsPTD1 gene is a gametophyte male sterility-related gene with a genome sequence shown in SEQ ID NO. 1 and specifically expressed in pollens. An interference element of an OsPTD1 gene was artificially synthesized at one time: an OsPTD1 self promoter (a sequence was shown in SEQ ID NO. 2)+an OsPTD1 gene interference target forward sequence (a sequence was shown in SEQ ID NO. 3)+a stem-loop sequence (a sequence was shown in SEQ ID NO. 4)+an OsPTD1 gene interference target reverse sequence (a sequence was shown in SEQ ID NO. 19)+Tnos (a sequence shown in SEQ ID NO. 5). A complete sequence Xi was shown in SEQ ID NO. 20, SalI+EcoRI were used for cutting a target fragment 2,935 bp from Xi to be recycled for later use, the SalI+EcoRI were used for enzyme-cutting the pA^{R}-Y-Bi-C at the same time, a skeleton was recycled for later use, and the target fragment and the skeleton were linked to obtain a final vector PA^{R}-Xi-Y-Bi-C.

### Example 2 Method for Breeding Recessive Genetic Male Sterile Line of Sporophyte

### 1. Breeding of Sterile Material Zhongjiu B-Osabcg15 of Sporophyte

A male sterile mutant (Wu et al., 2014) naturally generated by an OsABCG15 gene was used as a sterile donor, Zhongjiu B as a recurrent parent, and a sporophyte sterile material Zhongjiu B-osabcg15 was generated by multiple backcross.

2. Transferring of pA^{R}-Xi-Y-Bi-C into Zhongjiu B-Osabcg15

In a fertility-separated population, a sterile Zhongjiu B-osabcg15 plant was selected, young spikes of the plant were taken to induce a callus, the final vector PA^{R}-Xi-Y-Bi-C was transfected into the callus by a method of Agrobacterium tumefaciens, the callus was differentiated into seedlings, the seedlings took roots and were transplanted, 8 plants showing an anthocyanin color were selected as positive plants, and all the positive plants showed male fertility (FIG. 2).

### 3. Breeding and Purity Identification and Improvement of Genetic Male Sterile Zhongjiu B-Osabcg15 of Sporophyte

1) Breeding of sterile line

(1) seeds of a T₀ generation positive plant with a best fructification were collected, selfed progenies was planted, color and fertility were observed, the selfed progenies were averagely divided into two parts, and herbicides Basta (0.3%) and bentazone (0.24%) were respectively sprayed. Results showed ratios of colored progenies to colorless progenies, fertile progenies to sterile progenies, Basta-resistant progenies to Basta-intolerant progenies, and bentazone-resistant progenies to bentazone-intolerant progenies were all close to 1:1. Besides, the color and fertility were separated from Basta-resistance or bentazone-intolerance. The colorless property and sterility were separated from Basta-intolerance or bentazone-resistance. This indicated that all the transferred elements were functional. The positive plants were sporophyte-fertile, a capacity of generating pollens was recovered, meanwhile, a male gametophyte was sterile, and half of the generated pollens lacked a pollination capacity (with a transgenic component).

(2) The plants survived after spraying Basta were taken as a male parent to be hybridized with the plants survived after spraying bentazone to obtain 854 hybrid seeds. Meanwhile, selfed and fruited seeds of the plants survived after spraying the Basta were harvested. Hybrid seeds (as a female parent and named a sterile line) and the selfed and fruited seeds (as a male parent and named a maintainer line) were respectively sowed, bentazone was sprayed to the female parent to kill off-type plants in a seedling bed stage, Basta was sprayed to the male parent to kill sterile plants, an alternate planting was performed according to a proportion of 5 lines of the female parents and 1 line of the male parent, and an artificial auxiliary pollination was performed in a flowering period. Seeds on the female parent and the male parent were respectively harvested in a mature period, and 1.4 Kg of the sterile line and 0.8 Kg of the maintainer line were obtained.

2) Purity identification of sterile line

30 g of the harvested sterile line was sown in a mud tray. A pigment expression of coleoptiles of 1,000 plants was investigated. It was found that 969 coleoptiles were colorless and 31 coleoptiles were purple, and the colorless coleoptiles accounts for 96.9%. The plants after the color investigation were separately planted according to an existence of a color. Fertility in a flowering stage was investigated. It was found that all the colorless plants were sterile, all the purple plants were fertile, and purity of the sterile line was 96.9%. The results showed that a purple character was separated from a fertile character. The purple character of the coleoptiles at a very early expression period can be used for identifying the purity of the sterile line bred by the present disclosure.

3) Purity improvement of sterile line

Another 30 g of the harvested sterile line was separately sown in a mud tray to obtain 1,213 seedlings. Bentazone was sprayed at a 3-leaf stage. It was found that a few plants died completely after 14 days and 1,164 survived plants were transplanted. Fertility at a flowering stage was investigated. It was found that all the survived plants were sterile. After fertility investigation of 969 colorless sterile plants, and colored fertile plants which were separately planted for purity identification of the sterile line, the bentazone was sprayed at the same time. It was found that all the colorless sterile plants were insensitive to the bentazone and grew normally. However, the colored fertile plants were all withered finally. The results showed that the maintainer line mixed in the sterile line can be killed by spraying the bentazone, such that the purity of the sterile line was improved and the sterile line with a sterile plant rate of 100% was obtained.

4) Breeding of maintainer line: the seeds harvested from the male parent were sown. Basta was sprayed on a part of seedlings in a seedling bed stage. It was found that about half of the plants died and the survived plants were transplanted. Fertility and color in a flowering stage were investigated. It was found that all the plants showed male fertility and purple in parts such as leaf sheaths and the like. The other part of the seedlings was all transplanted. After the seedlings turned green, purple color-free plants were removed. Fertility in a flowering stage was investigated. It was found that all the reserved purple plants were male fertile. The results showed that the sterile plants can be screened by spraying Basta or according to an existence of a color, so as to breed a maintainer line.

In conclusion, this example indicated that the method designed by the present disclosure can be used for breeding a genetic male sterile line of a sporophyte, and can identify purity of a sterile line rapidly, accurately, and economically at an early stage, improve purity of a sterile line rapidly, thoroughly, and economically, and avoid rejection of a non-conformity sterile line.

The aforementioned examples are only preferred examples illustrated for fully explaining the present disclosure, and the protection scope of the present disclosure is not limited thereto. Equivalent substitutions or transformations made by a person skilled in the art on the basis of the present disclosure are all within the protection scope of the present disclosure. The protection scope of the present disclosure shall be determined by the claims.

1. A method for breeding a recessive genetic male sterile line of a sporophyte, comprising the following steps:
linking a sporophyte male fertile gene Y, a down-regulation expression element Xi of an endogenous gametophyte male fertile gene, a herbicide A resistance gene A^{R}, a down-regulation element Bi of a herbicide B resistance gene, an anthocyanin gene Cor a down-regulation element Ci thereof, and
transferring the linked genes into a sterile mutant of the sporophyte male fertile gene Y, and pollinating a sterile plant with a positive plant to breed a genetic sterile line of a sporophyte.

2. The method for breeding a recessive genetic male sterile line of a sporophyte according to claim 1, comprising the following steps:
(1) constructing a vector pA^{R}-Xi-Y-Bi-C/Ci containing the herbicide A resistance gene A^{R}, the down-regulation element Bi of the herbicide B resistance gene, the sporophyte male fertile gene Y, the down-regulation expression element Xi of the endogenous gametophyte male fertile gene, and the anthocyanin gene C or the down-regulation expression element Ci thereof at a same time, and transferring the vector into the sterile mutant of the sporophyte male fertile gene Y to obtain a male fertile transgenic plant of the sporophyte; and
(2) selfing the male fertile transgenic plant of the sporophyte, harvesting selfed seeds, dividing the selfed seeds into two parts, respectively spraying a herbicide A and a herbicide B, hybridizing a plant resisting the herbicide A as a male parent and a plant resisting the herbicide B as a female parent, and harvesting hybrid seeds to obtain a sterile line; or selecting a colored plant as a male parent to pollinate a colorless plant according to an existence of an anthocyanin color, and harvesting hybrid seeds to obtain a sterile line.

3. The method for breeding a recessive genetic male sterile line of a sporophyte according to claim 1, wherein the herbicide A resistance gene A^{R} is a Bar gene resistant to Basta; the herbicide B resistance gene is a Bel gene resistant to bentazone, and the down-regulation element Bi of the herbicide B resistance gene is an interference sequence of the Bel gene; the sporophyte male fertile gene Y is an essential gene, an OsABCG15 gene, for pollen development of paddy rice; the down-regulation expression element Xi of the endogenous gametophyte male fertile gene is an interference sequence of a gametophyte male sterility-related gene OsPTD1 gene; the anthocyanin gene C is a pigment expression related gene OsMYB76R; and the sterile mutant of the sporophyte male fertile gene Y is a sporophyte sterile material Zhongjiu B-osabcg15 generated by backcross using a male sterile mutant naturally generated by the OsABCG15 gene as a sterile donor and Zhongjiu B as a recurrent parent.

4. The method for breeding a recessive genetic male sterile line of a sporophyte according to claim 3, a nucleotide sequence of the Bar gene is shown in SEQ ID NO. 6; a sequence of the down-regulation element Bi of the herbicide B resistance gene is shown in SEQ ID NO. 18; a nucleotide sequence of the OsABCG15 gene is shown in SEQ ID NO. 17; the interference sequence of the gametophyte male sterility-related gene OsPTD1 gene is shown in SEQ ID NO. 20; and a nucleotide sequence of the pigment expression related gene OsMYB76R gene is shown in SEQ ID NO. 11.

5. The method for breeding a recessive genetic male sterile line of a sporophyte according to claim 1, comprising breeding a sterile line and comprising the following steps:
dividing the selfed seeds of a male fertile transgenic plant into two parts,
respectively spraying Basta and bentazone,
performing hybridization using a plant resistant to the Basta as a male parent and a plant resistant to the bentazone as a female parent, and
harvesting hybrid seeds to obtain a sterile line.

6. The method for breeding a recessive genetic male sterile line of a sporophyte according to claim 1, further comprising identifying and purifying a sterile line and specifically comprising the following steps:
after sowing a sterile line, investigating a ratio of a colorless plant according to an existence of an anthocyanin color to obtain a purity of the sterile line, and
when the purity is non-conformity, spraying a herbicide B to kill a colored and herbicide-intolerant off-type plant to obtain a sterile line with a sterile plant rate of 100%.

7. The method for breeding a recessive genetic male sterile line of a sporophyte according to claim 1, further comprising breeding a maintainer line and comprising the following steps:
collecting selfed seeds of a hybrid male parent,
sowing the selfed seeds, and
selecting a colored plant or a plant surviving after a herbicide A is sprayed as a maintainer line.

8. A transgenic progeny obtained by the method according to claim 1, or a plant material and a hybrid bred using the transgenic progeny.

9. The method for breeding a recessive genetic male sterile line of a sporophyte according to claim 2, comprising breeding a sterile line and comprising the following steps:
dividing the selfed seeds of the male fertile transgenic plant into two parts,
respectively spraying Basta and bentazone,
performing hybridization using a plant resistant to the Basta as a male parent and a plant resistant to the bentazone as a female parent, and
harvesting hybrid seeds to obtain the sterile line.

10. The method for breeding a recessive genetic male sterile line of a sporophyte according to claim 3, comprising breeding a sterile line and comprising the following steps:
dividing the selfed seeds of the male fertile transgenic plant into two parts,
respectively spraying Basta and the bentazone,
performing hybridization using a plant resistant to the Basta as a male parent and a plant resistant to the bentazone as a female parent, and
harvesting hybrid seeds to obtain the sterile line.

11. The method for breeding a recessive genetic male sterile line of a sporophyte according to claim 2, further comprising identifying and purifying the sterile line and comprising the following steps:
after sowing the sterile line, investigating a ratio of the colorless plant according to the existence of the anthocyanin color to obtain a purity of the sterile line, and
when the purity is non-conformity, spraying the herbicide B to kill a colored and herbicide-intolerant different plant to obtain a sterile line with a sterile plant rate of 100%.

12. The method for breeding a recessive genetic male sterile line of a sporophyte according to claim 3, further comprising identifying and purifying the sterile line and comprising the following steps:
after sowing the sterile line, investigating a ratio of the colorless plant according to the existence of the anthocyanin color to obtain a purity of the sterile line, and
when the purity is non-conformity, spraying the herbicide B to kill a colored and herbicide-intolerant different plant to obtain a sterile line with a sterile plant rate of 100%.

13. The method for breeding a recessive genetic male sterile line of a sporophyte according to claim 2, further comprising breeding a maintainer line and comprising the following steps:
collecting selfed seeds of a hybrid male parent,
sowing the selfed seeds, and
selecting a colored plant or a plant surviving after the herbicide A is sprayed as a maintainer line.

14. The method for breeding a recessive genetic male sterile line of a sporophyte according to claim 3, further comprising breeding a maintainer line and comprising the following steps:
collecting selfed seeds of a hybrid male parent,
sowing the selfed seeds, and
selecting a colored plant or a plant surviving after the herbicide A is sprayed as a maintainer line.

15. A transgenic progeny obtained by the method according to claim 2, or a plant material and a hybrid bred using the transgenic progeny.

16. A transgenic progeny obtained by the method according to claim 3, or a plant material and a hybrid bred using the transgenic progeny.

17. A transgenic progeny obtained by the method according to claim 4, or a plant material and a hybrid bred using the transgenic progeny.

18. A transgenic progeny obtained by the method according to claim 5, or a plant material and a hybrid bred using the transgenic progeny.

19. A transgenic progeny obtained by the method according to claim 6, or a plant material and a hybrid bred using the transgenic progeny.

20. A transgenic progeny obtained by the method according to claim 7, or a plant material and a hybrid bred using the transgenic progeny.


SEQUENCE LISTING (20 sequences)

SEQ ID NO: 1 - DNA, 2235 bp, Oryza sativa l.
atgacgacgacgacgcgagtggcggccgccgccgccggcgtgctgctggtggcggcggcgctggccggcgtggcgcgcggcgaggacccgtacgtgttcttcgagtggaaggtgacgtacggcaccaagaccctcctggacgcgccgcagaaggtgatcctgatcaacggcgagttcccgggcccgcggatcaactgctcgtccaacaacaacatcgtggtgaacgtgttcaaccagctggacgagccgctgctcttcacctggaacgggatgcagcaccgcaagaactcgtggcaggacggcctcgccgggacgcagtgccccatcgcgccgggcaccaactacacgtacaagtggcagcccaaggaccagatcggcagcttcttctacttcccgtcgctggggatgcaccgcgccgccggcggctacggcgggatcagcgtcgtcagccgcctgctcatcccggtcccgttcgacccgccggccgacgaccacatggtgctcatcggcgactggtacaccaaggaccacgccgccatggccaagatgctcgacgccggcaagagcttcggccgcccgcacggggtggtcatcaacggcaagtccggcaaggccgccgccgacccgcccatgttcaccgtcgaggccggcaagacgtaccggctccgcgtctgcaacgtcggcatcaaggcgtcgctcaacttccgcatccagggccacgacatgaagctggtggagatggagggctcccacacggtgcaggacatgtacgactccctcgacgtccacgtcggccactgcctctccgtcctcgtcgacgccgaccagaagcccggcgactactacgcggtggcgtccacgcggttcatccacgaggccaagtcggtgtcagccgtcatccgctacgccggctcgagcacgccgccgtcgccggccgtgccggagccgccggcgggatgggcgtggtcgatcaaccagtggaggtcgttccggtggaacctgacggcgagcgccgcccgccccaacccgcaggggtcctaccactacggccagatcaacatcacgcgcaccatcaggctcatggtctcccggggccacatcgacggcaagctcaagtacggcttcaatggcgtctcccacgtcgacgccgagacgccgctcaagctcgccgagtacttcaacgtcaccgacggcgtcttcaggtacaaccagatgaccgacgtgccgcccgccgtcaatggccccctccacgtcgtccccaacgtcatcaccgccgagttccgcaccttcatcgagatcatcttcgagaaccccgagaagagcatggactccgtccacctcgacggctacgccttcttcgccgtcgggtacgtacatacatcgccccccattactactacctccattaaccttttattaaagataggcttggccattcattttatttttaaaaaattatataagtatcatctattttattgtaatttgatttatcgtcaagtgtgctttaaacataatttgattttttttatatttgcataaaaaattgaatattatgaatgatgtatatgatctgatactactgttaactaacttactgatgcgcataataataaaattttttaataagacgaatgatataaaaaaagttttttaaattacctccatttataatatacgatgttttttttacttttaacattcgtttataatatatgttttttcacaaacgtttgagttttgaccatttattttatttaaaaataattatgcagttatcatctattttattgtaattttattgtaatttgattattgtcgagtgtactttaaatacgatttgatttttctatatttacataaaatttttaaataagataaatgatggatacgatctgatgctaccgttgactgactgactgatggcgcaggatggggccggggaagtggtcggcggaggagaggaagacgtacaacctgctggacggggtgagccggcactcggtccaggtgtacccgaggtcgtggacggcgatcatgctgacgttcgacaacgccgggatgtggaacgtgaggtccaacatctgggagaggcactacctcggcgagcagctctacatcagcgtcgtctcgccggcgaggtcgctccgggacgagtacaacatgccggagaacgccctccgctgcggcaaggtcgtcggcctgccgctgccgccgtcctacctcccggcctaa

SEQ ID NO: 2 - DNA, 1887 bp, Oryza sativa l.
gtcccatgtcaccgacagtactaaatgggtaaagattggataaagtatatggggtatttgtgaggtattattagaaaacttcgtgtggttttgatggacctgttttatgtgttgaaaatatgaatggttatagggtgtgtttgcaagtgcaggatgggaactcatccctcctgcacgcaaaacggagcggctttttaacacatgattaattaaatattagctaatttttttaaaaaaaatggattaatttgatttttttaagcaactttcatatagaaattttttgcaaaaaacacaccgtttaatagtttaaaaacgtgcgcgcgaaaaacgagggagaggggttgggaacatgggtttgcaaacacaaccatagtattggcgattccttttcgtttgagtaaattttacaaaactacaggtattttgaccaaattatcacaaaactacagatttaaggagttgtatcataaaactacacatttagcatcaaatttatcacaaaactgcagattttaggttaagtatcacaaaaatacatatttaatattgaacttatcacaaaactataacttttggagtttaaatccctagcaccattgttatggtggagctataaacattattactttgtgattaaattggttctaaacctttagttttatgataatttagtaactaaacgtgtagttttgtaacacttcatctttaatatgtagttttgtgctaaatttggtgctaaatgtgtaattttgtgatataattccttaaatatgtagttttgtgatagtttggttataatatctgtagttttatgaaatttactcttttcgttttcactgcaatttggaatgatggaattgactagatccggcattaccgatgggctgccgaacgctgtgatgcggttgatcttgagcgatccgggacgccacaagcaccgatgggttctgggagttcatacggctggtgcagcagtgtgtcaatagcagccgggatgtgcgcccaaccatggtcgccgtcgagaggaggatcgaagacatcctgaactcggttgtcaggtcatccaccaccgggttcatgactgccggaggcgacacacccagcaacgagccaaatcgtgaagataacggaaacgagccaaatcccagcaacgagatcgccagggactagtagtacgtacagcagtggtgatttgtcatataggtgtatatcggctgttttcgcatctcaaggcctcaagcagtgtgtgcaatctggagtagtatataaatatgtaaaatgttcatttcgatatactgtcaaatgcgtgtaaattaaccaatgctaaaacaacacactgtgactaaatttactgagttggatgatgaggatgattatgttgcgtgcacacctgatcaggaggacatataatataggccatttgggccgtcttggacaccaccgtttgatttgtatgaagttgggccgaactatgcaagcccagaggcgctgcctctgtgccacggcccacgggcatcgctggatggtcaagcaggtgatcggtggagcgccaatggcggcggcgagacacacagcgcggcgcgcgcgcgaacgtgcggacgcgcgcgccccggccacggccgccgcgctcgtctcctggcctcccgcgcccgctacaaatggcggccccggcgtcccctcctcactccgaagcttcccggttgacgacctctccggtctcccccctcaccccaccgcaacccgggacgtcttccatggccgccgccgccgccgcccccgcctactaaaccaccctacccaccccctccaaactcccacacattacatccttcaaagagagcatcacacacacacacacaccagcctagcgatcacatttccacg

SEQ ID NO: 3 - DNA, 311 bp, Oryza sativa l.
gcagaaggtgatcctgatcaacggcgagttcccgggcccgcggatcaactgctcgtccaacaacaacatcgtggtgaacgtgttcaaccagctggacgagccgctgctcttcacctggaacgggatgcagcaccgcaagaactcgtggcaggacggcctcgccgggacgcagtgccccatcgcgccgggcaccaactacacgtacaagtggcagcccaaggaccagatcggcagcttcttctacttcccgtcgctggggatgcaccgcgccgccggcggctacggcgggatcagcgtcgtcagccgcctgc

SEQ ID NO: 4 - DNA, 150 bp, Oryza sativa l.
acgagctggtgagctagctattacctaatcgatcgatggtcatcgatcatgagatgatgatgatgagatttgtacttaattgtgatctgtatggatgctgttgttgatcaagttcttgcgatcgatcgatctgaattttcaggtttgagg

SEQ ID NO: 5 - DNA, 270 bp, Artificial Sequence
  Synthetic
gctcgaatttccccgatcgttcaaacatttggcaataaagtttcttaagattgaatcctgttgccggtcttgcgatgattatcatataatttctgttgaattacgttaagcatgtaataattaacatgtaatgcatgacgttatttatgagatgggtttttatgattagagtcccgcaattatacatttaatacgcgatagaaaacaaaatatagcgcgcaaactaggataaattatcgcgcgcggtgtcatctatgttactagatcggg

SEQ ID NO: 6 - DNA, 1254 bp, Artificial Sequence
  Synthetic
gaattcctgcagccaccatgttggtgagatttttcaaatcagtgcgcaagacgtgacgtaagtatccgagtcagtttttatttttctactaatttggtcgtttatttcggcgtgtaggacatggcaaccgggcctgaatttcgcgggtattctgtttctattccaactttttcttgatccgcagccattaacgacttttgaatagatacgctgacacgccaagcctcgctagtcaaaagtgtaccaaacaacgctttacagcaagaacggaatgcgcgtgacgctcgcggtgacgccatttcgccttttcagaaatggataaatagccttgcttcctattatatcttcccaaattaccaatacattacactagcatctgaatttcataaccaatctcgatacaccaaatcgaatcgatgagcccagaacgacgcccggccgacatccgccgtgccaccgaggcggacatgccggcggtctgcaccatcgtcaaccactacatcgagacaagcacggtcaacttccgtaccgagccgcaggaaccgcaggagtggacggacgacctcgtccgtctgcgggagcgctatccctggctcgtcgccgaggtggacggcgaggtcgccggcatcgcctacgcgggtccctggaaggcacgcaacgcctacgactggacggccgagtctaccgtgtacgtctccccccgccaccagcggacgggactgggctccacgctctacacccacctgctgaagtccctggaggcacagggcttcaagagcgtggtcgctgtcatcgggctgcccaacgacccgagcgtgcgcatgcacgaggcgctcggatatgccccccgcggcatgctgcgggcggccggcttcaagcacgggaactggcatgacgtgggtttctggcagctggacttcagcctgccggtcccgccccgtccggtcctgcccgtcaccgagatttgactcgagaatcttggactcccatgttggcaaaggcaaccaaacaaacaatgaatgatccgctcctgcatatggggcggtttgagtatttcaactgccatttgggctgaattgaagacatgctcctgtcagaaattccgtgatcttactcaatattcagtaatctcggccaatatcctaaatgtgcgtggctttatctgtctttgtattgtttcatcaattcatgtaacgtttgcttttcttatgaattttcaaataaattatcagatccggtaccgagctcaagctt

SEQ ID NO: 7 - DNA, 8543 bp, Oryza sativa l.
gaattctagtttggattccctctctaaccttttcgtggggttaaccgttaaattcatattaaaaagtccatttttcctttcagtatagaggaacatatatatatatatatatatatttgtgagtttgttatacgagatattatttatgacaaaatttatttaaaaatatgatatagaaaataatatttatttatctgttattaaaaattgtttacgtaggatataaatagatttgtcttatactacaatcaaaataatacagtacttatataatttttaataactaaaaaaataaatattattctttcatgtcatatataaaaataaattttatcgtaaataatatttttcataacaaatacataaatataaaagtctcgtacaataaatttttatactccaataatatactcataccaaaaggtaaaatggacttttgattaagaatttaatggtcaactaacggaaaagttatgaaagggatctaaactaaaatttagaagtatgcaaggaaataagcaaaacttagtaccacataagagattagacaaagtcttataggtacataagaaatttctcttatttttgggaagcactagtagtgtcgtttgtggagtcgtaggtgcatgactccacccaccatgctttaaatcctgatgcccacgaatattacgcacatgcatgtgaactttcaataggactttagtgagacgctggtttccgtctctaagagcatgtgttaggagacacatttgttggggtgtgagtatggtgttacgtgtgtagtggtgtgtgtctgcggtataatcttaaaaaaaagttctcttatttttgtggtgcataatttttgacatggaagacctttttctcgtcacagacaagcaccaccgggcaccgactgatttctgaagtcgtcactggttgtccctcgtgaatcgtcgtcacctgctaagcccaaatatccacagaaagttagtggtatcaaattatgttatctccattcgatattttgggtttattttaagtcaaatatattaagtttgatcgagtttgtagaaaaacgtagcaacatctataacatcaaattagttttattgaatccacgattgaatatgttttatagtatatttgttatgtgttaaaaatattgctatatttttttataaattttagtttgacttatgacaaatccaaaatgttttacaatatacgcgtatgacaatctttgaggtgtaggagattaaattctttgcatttgattgtttatacatctagttttgtcacatgaaataacacacagttctgattttcaaagaaaaaaagtaacagataattctgatgtgcatacatggaaggttctcttctgaaagcccttagaaagattgtgggtcaccaaacaaggtgcagaaaaaaaacaagaacacaacagtatatatatataacaaagtaggcatgctcttggttggttactagtggttctgtttctcatgctcatccatgcatttcttgctacctgattaaaaggagagaagggcagtctgcttggcgctctctctctgaacctgtcactctttttttctgtgtctcttggaggagatgatggagatcagcagcaatgaggagatgatggagatggccattgttgagcagctgcctccttcctctcatcatctcaatggtggcagtgttgaggttgacatggaggaggatcatgtgtggccaaccaaagatggccctctccctatattccttaaggtgtgtatatctatctcttcttccatttttcggtatgcgcgaaaacagatcaagtttatccttgttgatgttcaagaaagtgttttaatccctcgtttatggttatgaaaaattatgaaaacaataaatagattatccctataagcttttataggtaagtggatacatatgtgcatgtttgtaggtatgtgaaaagattaatcagaatgcttacacatgttgatgttctccctatgtagtgtgtgtatatatgtgcatgtttgatgtttgtagcaactgaacaatttagttctactaagcagagtttgaaaaagatcgatattactaccaggatttattaagatactaaaggttttaccgtgattaatcatgaaatcatgaaaaaaagtttgtattaattatttcttatgcattttatagagtctaattttgaactcagagatgcaaaaaatgccaaacaaaggaaacgacctgaaatagacatacttaaggagggaaggtacaagttagttggttggcaatttgcatgtctcattgctcaacagtttcttatctaattgcaaaaacttcttgcatggtgtttttacttctttggcatgcaatatagctaacaaaaaaggagcaaattcctggtctaaatttttgcttgatatataaatatttttttttggaaagtgcagtttgagaatgtggagtacaaggtgaagctgactccaaagaacccactaacagctgcaagggtggcatttgcatctcacaagagcactgaggatcagggcagctgcaagcacatcctcaaggggataggaggcagtgttgaccctggtgagatcctggcactgatgggcccatctggcagtggcaagaccacgctgctcaagatacttggaggcaggctcagtggtggtgtcaagggccagatcacctacaatgacaccccctacagtccctgcctcaaaagaaggtattccctctatttcatattataagtcgttttgactttttcttatttaaacattattaagtttaactaagtttataaaaatagcaatgttttaaacaccaaattagtttcattaaatttagcatcgaatatattttgataatatgtttgtttaattcttttgtgttaaaaatattactatatttttctataaacttacttaaacgtaaagaaatttaactagaaaaaaaagttaaaatgacttataatatgaaatagagagagtaactgtataaagtcatagtggatattaattagtatgatttatgtaatgagaaaggtccggggtcttccgactagcacgataaggtgtgggttagttgatcaagttcaagcctcatctctcttaataaattttgatataaaagttatttctctcatatctagtgtttttttttatttatggtagttattgtaagaccataaggtatgggttaggccctgtttagtttccaaaaacttttcgaaaaaacatcacatcgaatctttagacacatgtatggatcattaaatatagataaaaataaaaactaattacacagtttgtatgtaaatcgcgagacgaatcttttgagcctaattagcccatgattagccataagtgttacagtaactcacatgtactaatgccggcttaattaggctcaaaacattcgtctcgcggtttggcgagttatgaaattagttttttcattcgtgtccgaaaaccccttccgacatccggtcaaacgtccgatgtgacacccaaaaattttcatttcaccaactaaataggcccttagttattgtaagatagaatttttgcatagttatgctatctttcgtaaactagtggagtagattgagtaaaaccagtatgtcttcaaagagaatttacaggcacgcaaatgactgacaaactgcttagtgtttactgcaatcttcagataagagagaacatgtattggcatgccaaattaagcagtgtagttgcattgttctgcaggtatcaattaaagtggaaaaattaagtaatcaagactaattattttcttttgatgaaaacaggattggatttgtgacacaggacgacgtccttttccctcagttgacagtggaggagacccttgtgttcgctgccttcttgagactccccgctcgcatgtcaaagcagcagaagcgcgacagagttgacgccatcataactgagctaaatctagagaggtctgtaagtatattttcatcaaaggaaaaaaaaaactacaccgattttactaatataagttgtcaaaccaacaatctgcagcttaacacaaacccacgtgaagcaaaatagcatacaaaataaaatcatgactaatagcataaatctgttttttttctgaaattcgaataagaaaatgcaaagaaacgcaaatatcgcatcttaaagtacagtggtgcattagaattactcttgatttttctcgaccaacctacatgacatacctactgaacgtgcactttcatttcaaaagagaaattttacagttcttaaagagttattatattttgtagtgcaaatcaaaatttacattagaaaaaaaatgatatcttttaacggtcacaaaattactcatcttaaaaatggtgggtcatacggcaatggtatacccattgaacagtgtttacgatttcggaaaattttggaccctggcacaaaactacttcactgaattatttttcccttttttttcatgaatttgatcgaaaatttgttcaaaacctaataaatagtttttaaatatccaatatctcggaaaatttcgctacccaggacccacgacacaaatgctgaaaacgaaaaggactacctattaaacttttggtacatttataactttgaatcttacataattttcaactaatagatatatagcccgttttaaatgaaatagaaaaaaaagagacaaacatgtcaaaaaaaaaaccgtaaatctaacttaaaataaaatgttgtgaaaaaaattctaatgtttgtacacgattgcaaaactctgcaggtgccggcacaccaagatcggcggcgcgttcgtgcgcggcgtgtcgggcggcgagaggaagcggacgagcatcgggtacgagatcctcgtcgacccgtcgctgctgctcctcgacgagcccacctcgggcctcgactccacgtcggcggcgaagctcctcgtcgtgctccgccgcctcgccaggtcggcggcccgccggacggtgatcaccaccatccaccagccctccagccggatgttccacatgttcgacaagctgctgctcgtcgccgagggccacgccatctaccacggcggcgcccgcggctgcatgcgccacttcgccgccctcggcttctcccccggcatcgccatgaaccccgccgagttcctcctcgacctcgccaccggcaacctcgacggcatctcctcccccgcctccctcctcctcccctccgccgccgccgcctcgccggactcccccgagttcaggtcccacgtcatcaaggtatgtgcgtgtctcgtgtcgagggcccaagaatcactctcatggcgttgtgggtgcagtacttgcaggcgaggcacagggcggccggcgaggaggaggcggcggccgcggcggcgagggagggcggcggcggaggtggcgcggggagggatgaggcggcgaagcagctgagaatggcggtgaggatgaggaaggataggcggggcggcatcggctggctggagcagttcaccgtgctgtcgcggcgcacgttccgggagcgcgccgccgactacctcgacaagatgcgcctcgcccagtccgtcggcgtcgccctcctcctcggcctcctctggtggaagtcccagacctccaacgaggcccagctccgtgaccaagtatgtacactagcttcttcttcctcctctcgacaccgccattgctgacgtcatcaagaactcaaactgtccgatcaagaacacatacacaaacccaccctctccttcttcctcctcctctcgacaccgccattgctgacgccattgtcaagaacatacattcctcttcttcctcctcctctcgacaccgccattgctgacgtcatcaagaactcaaactgtccggtcaagaacacacaccctcttcttcttcctcctctcgatatcgccattttcaagaacatacactcctcttcttctttctcctcctttcgacaccgccattgctgacgtcatcaagaactcaaactgttcgatcaagaacatacacataaactcctcttcttcctcctcctctcgccaccaccattgctgacgtcatcaataactcaaaacacacacctcttcttcctcctcctcctctcgacaccaccattctcaagagtcaagaacatacactcctcatcttcttcctcctcctcctccttgcacagttgcaccgccatcggcaagaagtcgaggtgatcaagagtaatatgtgtgcgtttgcaggtggggttgatattctacatctgcatattctggacgtcgtcgtcgctgttcgggtcggtgtacgtgttcccgttcgagaagctgtacctggtgaaggagaggaaggcggacatgtacaggctgagcgcctactacgcgagcagcacggtgtgcgacgcggtgccgcacgtcgtgtacccggtgctcttcacggccatcctctacttcatggccgacctgcgccgcaccgtgccctgcttctgcctcaccctcctcgccaccctcctcatcgtcctcaccagccagggcaccggcgagctgctcggcgccgccatcctcagcgtcaagcgcgccggcgtcatggcctcgctcgtcctcatgctcttcctcctcaccggcggctactacgtccaggtgaatctcacaaatatcatcttctgatttctgaattttggttctcgatcactttcccgtgttctaaaacatggtttaaatgttgaagaaatcaaaagtattttggaaggacggatagaagtgaaattttggactagaaagcacaaaattatcttcttctagtttctgaattttgcttatcacaaacatggttataatattgagtaaattagaaacatttcttcttttagaaaaaaaaacaaaaagtatttcagaaacagagtttgaacaagcaatttgtttggctgaatttaaacatgttctactcaaatttaaaaaaaactatttttttcggttaaaataatatctgggaggccatatacccaaaatttctaaaattcctttccgaaaatttcaaaaataaatttgctccgaaatgtgaacgaacagggccaaaaaaaacacacaaaatattacatatcattgtgcatgcatcgactatttatcgacattatattcctaacttttttcacaccaaataagctcggataaaaatgaaattgaaatttgggataattttgtttggattgaacaaaaaacattgcagcatataccgaagttcataaggtggctcaagtacgtgtcgttcatgcactacgggttcaacctgctgctgaaggcgcagtaccacggccacctgacctacaactgcggcagccgcggcggctgccagcggctgcagtcctcgccgtcgttcggcaccgtcgacctcgacggcggcatgcgcgaggtctggatcctcctcgccatggccgtcgcctaccgcctcctcgcctacctctgcctccgcaagcggatcagcctcatgcccttgtagtccatcgatctgtcagatcaggatcagttggttcggggaagaagacgactgatgtggtagtgaattgcaattgcgattgatgatggtgaatgggccattgttggtttgggtgctgtgaattgtttcaaacttgagttcttttctttatttgagaagtatttgaggcttgtctgcttagcaagttgggaaataatcaaccccatcattttagcattcagcttgtaagcataagcgaagatttatttaaaacttatttttaaagttaattttaggtatcttttcgtagtttattttctagcatttactttttaatttgctgagcatgtatattaaagttttacgtataattttttttaaaattattaataagttgtttgtataagcgattagtctgtctcgtcgtcgtgacaaaaagaaaaaccgatgagatggttgggagccgattaaggttattgttttcctcgaaaagagaaggtggaggggatggggtgaatttaaatctccctgcaaaaccatttttggctttgagctcaaaatttctcattttccgtttgtaatggacatagtagtatgttctcctcagggcctttgccctattaggcccaaatctcctgcccgcaaaaaatgggctgacaacgtcacgaaccacgaaccgcgccgtaaatgggccaaaaataccaaggcccaaatcctacagagcccaaagtagagggccataatatgggcctagaaattcaacggcccgctccaatgagaagtggactcggagcgggattcccccaccaaatccgtttcgacctcagaaaatacgcctccgatcaggcgtttacgcacactactaatattgacgagaaagccacctcctctctctcgctcgctccgctcttcttgacaaggaagcaaaagccgccgccgccgacgccgacgagagaggagagagagatcgccgccgacgtcgcaggagggggagggggagatggcggcggagggggaggacgcggcggccgcgcgacgccgcgcggcggccaccgactacaggaagaagctcctcacctgcagg

SEQ ID NO: 8 - DNA, 6592 bp, Oryza sativa l.
gatcacgacctatgtaatcaacctgttgtgttgtgagcgtgtgtgcctgatttggttgcctgcaggtgttgtcctggccaccgacttcgaagcaatctgtgcgccggttggaggtggcggagcactggtaccgactctacaagacggacaatcaacgggtaccacaatccggctccacctcgacgcgattgcggcaaccacgaaaacctcacgctccaatctgtgccctccgcccgtgctcgctgcatctcgtgccaccgtctcggactctcgatcctcatggcctgaattatcctaattcttcgttaccagtttttttgagactaatatgactcccatcaaacaatgcagttgagagtgagttcttacctgtattatatagtacattgtatttaaaccatagtacatggggacagtggtgcgttcatcaatttatggattgtggtaggctggtagctgtttgatttgtcactaaaattgcacgacgacaacactttggccctgtttagattctaacttttttcttcaaacttccaacttttccgtcacatcgaacttttctacacacacaaacttccaactttcccgtcaccttcactttaggttcctaagtttaggttaccttcactttaggttcctaaatttatcactaagtctgaaatttatccctaaaccaaaataccaggtacaacggatccctcaatctacaaaactcaatcacccaagattgtagatagtattatgtccggttttaactgacgtgtcaagttgagttaacgtggaatctatgcgggccccacatgtaagtggctagtacttttttccacgttggacgaaaccgtctcccaaaccactaaaggaggcgatatgcaccgattttgatagatgggggagacgttataccctgttttattatcaagagatgtgattcaaccaggagcaagagttgagagagcaagaatagacttattcgtgagcccacaggctaaacccgccacggcccacagactaagcccgccaactcgggccgttcggcccagtttgcacgcatggtgggccggaaactggaatctccgaaccgcaacggaatcgacgacatctgtagccacagcgcgcactcgacggcgggcagcttcgtcactggttcgagctgtctgctgactcaacgacgaacacacgtactcctgctcggtttcttcctccgtgtcgcacaaaagtcaaattgctctctattagtattcattattagcacttcaaacctttctttacgttttaaacgaaccaactaatcaggttatgaggactataataatcgaatccagggatcttgctggaagcaattgaataatcgcccaacgattgagttcatttcttgtctccaaagctgtctcctgatagtcaacaggtctcggtcctcgcagagtcgcactgcgatttggcctctggaaactggaaagagatctcgatccaccacaagaaaatgccaagcagcacgacgacgaagacgacaacgccttccaggtacgtagcaattgaatacggaatactctctccgtttaaaacgtttgattattttcctaatcaaactttatcatgtttgaccaaatttatagaaaaaaataacaacatcttaaatataaaattagtataactaaatctagcattggatatactttcataatatttgtttgttttatgttaaaaatactactatatttttctataagcttagtcaaatttaaatcaaattaaagaagattaattagaaaaatagccaaacgatttgtaatatgcaacggagtgagtagaagtaatcgcccagcctcgccaacgaggcaacgagacccgtaatgcaacgatcgcatctgcgtttcaggcgtcagccatggcgtctgcagagatgcctggattgtctccgcaagatctgatccatttcatctccttctagaagcacaagcgccgctcggtataaaggcagacgcattgtcacaaatagctgcagtgcaccagagtcacagaaacacatcacacattcgtgagctcagcttagccatggataacgcctacattattgccattctctctgtagctatcctcttcttgctccactactacctcctcggccgcggcaatggcggggcggcgcggctgccgccgggtccaccggccgtcccgatcctgggacacctccacctcgtcaagaagccgatgcacgccaccatgtcccgcctcgccgagcggtacgggccggtgttctcgctgcgcctcgggtcgcggcgcgccgtggtggtgtcgtcgccggggtgcgccagggagtgcttcaccgagcacgacgtgaccttcgcgaaccggcccaggttcgagtcgcagctgctggtctcgttcaacggcgccgcgctcgccacggcgagctacggcgcgcactggcgcaacctccgccggatcgtcgccgtgcagctgctctccgcgcaccgcgtcggcctcatgtcggggctcatcgccggcgaggtccgcgccatggtgcggaggatgtaccgcgccgcggccgcgtcccccgccggcgccgcgcgcatccagctgaagcggaggctgttcgaggtctccctcagcgtgctcatggagaccatcgcccacaccaaggcgacccgccccgagacggacccggacaccgacatgtccgtggaagcccaggagtttaagcaggtcgtcgacgagatcatcccgcacatcggcgcggccaacctgtgggactacttgccggcgctccggtggttcgacgtgttcggcgtcaggaggaagatcctcgccgctgtaagccggagggacgcgttccttcgccgcctgatcgacgcggagcggcggaggctggacgacggcgacgagggcgagaagaagagcatgatcgccgtgctgctcactctgcagaagacagagccggaggtgtacaccgataacatgatcacagctctaacggcggtgagttcatcttctgctgttttacctttctgatatctgaattctctcattggtgcgtaattttttttttttggctgtcatcggtatagctttcttaagcactcagtagccttgcaattataaaaagaaaaacaatcagtagctttttacatgctttgagtcagtcagtagcagtgtggcactatcagcattcagcagtattcatgttgtttgctaatcactatcatggtttgagtcagcacaatcagtagcttttgacatggtttgagtcagcagtatcttttctaggaactgaattagttattcatttagtacaacttgtttgtctgtctattgattgctttaaattatttcttctatgcaaccctctaatcctagtatagtactagccttttatatgaagaatcatcaataattttcttctcactttcagtgtagctttacttaatgaatattttgaaagatcgcctagttgccttattataattgtataaaaggaaaacaatcagtagccttttacatggttgagtcagcgagttatcaggagtactattgtttatcatggtagtagcacgatagactattcaacccgggcgattaaaatccttctcccaaatttgtattctttgttttttcctcaaaaattcaccaaactcttggaactattatagttcagttttagacaaaaaaaaaatgaaatcttagttccaccctcttgccgtgcagaacttgttcggagcaggaacagagacaacctcgacgacatcagaatgggcgatgtcgctactgctgaaccaccccgacacactcaagaaagcgcaagccgagatcgacgcatccgtcggcaactctcgcctgatcaccgccgacgacgtgactcgcctcggctacctccagtgcatcgtcagggagacgctccgcctgtaccccgccgcgccgatgctcctcccgcacgagtcctccgccgactgcaaggtcggcggctacaacatcccgcgcgggtcgatgttgctcatcaacgcgtacgccatccaccgtgacccggcggtgtgggaggagccggagaagttcatgccggagaggttcgaggacggcgggtgcgacggcaatctcttgatgccgttcgggatggggaggcggaggtgccccggcgagacgctggcgctgcgcacagtggggttggtgctgggcacgctgatccagtgcttcgactgggagagggtcgacggcgtggaggtcgacatgactgaaggtggcgggctcaccatccccaaggtcgtgccgttggaggccatgtgcaggccgcgcgacgccatgggtggtgttcttcgcgagctcgtctgaatattttttggcggcgtttgcatctccaggacgaactcatgtattgaaagcaccaaaagtaagtagcaaataagcttctcgtgagcatacacataacacatgtgagcttgtaatgtggaataaattacacgtagaggatttggaagagagtgactgcgctagcaatcgctctttgagagttgtgttttacagttttagtgaggaaccaatttgtatgaatgtgcaataatcatgtataaagtataattgtacacgccaggtatttcaatttcatattgcttgtgtgatgtatgcgttcgaatactatacagtcaacaaaagagaacaaaattactatgagcttatacgaccagtgttcaaaataatagccagtcaatggtcaataaaaattaaaattaaaaaaaaagtcttacaaattcctgattttctatcgtttgaaaaaaaaaaacaacttcgggatattacacatattacatattgatccaataatcgaatatttcaaatactttcttaacgacttctaacttgggaggaagggctttctgctctttggctcctgtccatgcagacaagcagacaacacaagcaaaaggggaaaaaagttcatttacatccctcaaatatatgtcgagtttcaattgcgtccctcgatcgcataaccagatatagagcaccgttgacttgtaaaaccacgtcagctagaaaagcattcatccaaaaccgccaaggaccatactctctccgtttcaggttttaagacgttttgactttagtcaaagtaaaactgtttcaattttgactaagtttatagacaaatatagtaacatttataatactaaattagtttaatcaaatcaataattgaatatattttcataataaatttgtattgggttgaaaatgttactacttttttctacagatttggtcaaacttaaagcagtttaactttgaccaaagtcaaaaacattttataacctgaaacgaaggtagtactttacactgattttacatgctacggatgctccatatctgatttttatacttgagggattcgattcaaacacgattcttgttgtgagggacctaaagtgaaccaagtaaaatgcgttcatcttttgcatgctttcatgggtcggggcctcggggagagaaacctccgagccccacggcccacaggcctaccccgccaattccaatcgttcggcctagaaatctccgagccccacggcccacgtcggccaccgatgaattgtggccggcgacaaccatctctcatcccctctcgacggacgtcatcggcgttccttgaccttgtgggtccagatcccctagagttgggtataactctaccgggtagagttggccaaatctgtgccattcggcatacgtcaagggctgggattaagccacattaatttatacttaaaaccagtaactgaccatgcaacaaataaacttacatcaacagcttgtcacgagatcattgttactgtataattagcacaaaaaaaattcaagattcatctattatttaaaaaatcatacacattattattgatatggttcgttgtttgcatatatgtgaaagcactatattgatatacactgactgccgctttagagttgatggactatatctgttgtttggggaataacaaaacgttgttctgttacattttttgggagtgagaacttaatcaaatcactattatagagcagacacatgaaaaaactaagtcataaccattaatttaaggagttaatttattaggtgaaatttcctttaaaaaatttattaggttaaattgatgaggaatgatctcagccgtagaaaacaatagatagcacagatttgactaacttcacccggtagagttatactcaacttcaacttcaggggatccagacccgacctttgtgctctgctagcaaattaaccgggggtgaggatttgctctaatccattgatcaatcatcgacacctcacgttacaaacgtgcacggattctttttctgaataaaaccaagcaactacgaggccaaaacatcgtactccatgatccatccgatgcagatatccaattattggactccatgacagcatgcaccgatcgcttccatagacgacgtcacagatggcgcacgcaacactgtagcactgtaccacagcgatagactagctctatctaccgctggggaaagtgatttcatcgctcgtgagaataacatctcgtttcgttcatggcatctaaatagtcataaaagtttt

SEQ ID NO: 9 - DNA, 772 bp, Artificial Sequence
  Synthetic
gcagaaggtgatcctgatcaacggcgagttcccgcgcccgcggatcaactgctcgtccaacaacaacatcgtggtgaacgtgttcaacctgctggacgagccgctgctcttcacctggaacgggatgcagcaccgcaagaactcgtggcaggacggcctcgccgggacgcagtgccccatcgcgccgggcaccaactacacgtacaagtggcagcccaaggaccagatcggcagcttcttctacttcccgtcgctggggatgcaccgcgccgccggcggctacggcgggatcagcgtcgtcagccgcctgcacgagctggtgagctagctattacctaatcgatcgatggtcatcgatcatgagatgatgatgatgagatttgtacttaattgtgatctgtatggatgctgttgttgatcaagttcttgcgatcgatcgatctgaattttcaggtttgagggcaggcggctgacgacgctgatcccgccgtagccgccggcggcgcggtgcatccccagcgacgggaagtagaagaagctgccgatctggtccttgggctgccacttgtacgtgtagttggtgcccggcgcgatggggcactgcgtcccggcgaggccgtcctgccacgagttcttgcggtgctgcatcccgttccaggtgaagagcagcggctcgtccagcaggttgaacacgttcaccacgatgttgttgttggacgagcagttgatccgcgggcgcgggaactcgccgttgatcaggatcaccttctgc

SEQ ID NO: 10 - DNA, 1975 bp, Artificial Sequence
  Synthetic
ccaccgacttcgaagcaatctgtgcgccggttggaggtggcggagcactggtaccgactctacaagacggacaatcaacgggtaccacaatccggctccacctcgacgcgattgcggcaaccacgaaaacctcacgctccaatctgtgccctccgcccgtgctcgctgcatctcgtgccaccgtctcggactctcgatcctcatggcctgaattatcctaattcttcgttaccagtttttttgagactaatatgactcccatcaaacaatgcagttgagagtgagttcttacctgtattatatagtacattgtatttaaaccatagtacatggggacagtggtgcgttcatcaatttatggattgtggtaggctggtagctgtttgatttgtcactaaaattgcacgacgacaacactttggccctgtttagattctaacttttttcttcaaacttccaacttttccgtcacatcgaacttttctacacacacaaacttccaactttcccgtcaccttcactttaggttcctaagtttaggttaccttcactttaggttcctaaatttatcactaagtctgaaatttatccctaaaccaaaataccaggtacaacggatccctcaatctacaaaactcaatcacccaagattgtagatagtattatgtccggttttaactgacgtgtcaagttgagttaacgtggaatctatgcgggccccacatgtaagtggctagtacttttttccacgttggacgaaaccgtctcccaaaccactaaaggaggcgatatgcaccgattttgatagatgggggagacgttataccctgttttattatcaagagatgtgattcaaccaggagcaagagttgagagagcaagaatagacttattcgtgagcccacaggctaaacccgccacggcccacagactaagcccgccaactcgggccgttcggcccagtttgcacgcatggtgggccggaaactggaatctccgaaccgcaacggaatcgacgacatctgtagccacagcgcgcactcgacggcgggcagcttcgtcactggttcgagctgtctgctgactcaacgacgaacacacgtactcctgctcggtttcttcctccgtgtcgcacaaaagtcaaattgctctctattagtattcattattagcacttcaaacctttctttacgttttaaacgaaccaactaatcaggttatgaggactataataatcgaatccagggatcttgctggaagcaattgaataatcgcccaacgattgagttcatttcttgtctccaaagctgtctcctgatagtcaacaggtctcggtcctcgcagagtcgcactgcgatttggcctctggaaactggaaagagatctcgatccaccacaagaaaatgccaagcagcacgacgacgaagacgacaacgccttccaggtacgtagcaattgaatacggaatactctctccgtttaaaacgtttgattattttcctaatcaaactttatcatgtttgaccaaatttatagaaaaaaataacaacatcttaaatataaaattagtataactaaatctagcattggatatactttcataatatttgtttgttttatgttaaaaatactactatatttttctataagcttagtcaaatttaaatcaaattaaagaagattaattagaaaaatagccaaacgatttgtaatatgcaacggagtgagtagaagtaatcgcccagcctcgccaacgaggcaacgagacccgtaatgcaacgatcgcatctgcgtttcaggcgtcagccatggcgtctgcagagatgcctggattgtctccgcaagatctgatccatttcatctccttctagaagcacaagcgccgctcggtataaaggcagacgcattgtcacaaatagctgcagtgcaccagagtcacagaaacacatcacacattcgtgagctcagcttagcc

SEQ ID NO: 11 - DNA, 819 bp, Oryza sativa l.
atgggacgcagggcttgctgtgcaaaggaaggaatgaagcggggtgcctggacgtccaaggaggacgatgtcctggcgagctacattaagtctcacggagagggtaagtggcgggaggttccgcagagagccggacttcggagatgcggcaagtcctgtcgccttaggtggttgaactatctccgcccaaatatcaagaggggcaacattgacgatgacgaggaagagctgatcgtgcggcttcatacgctcctggggaatagatggtccctgatcgcaggccgccttccgggaaggaccgataacgagattaagaactactggaatagtacactctccaggaagatcggaacggccgcgaccgccgccgccggctcacgcggcggctccacccctgatacagccagggcgactgacgcggcttccagctcttcagtggtcccaccaggacagcaacagcaaccagcaagccgggcggatactgacaccgcaaccgccgccgccgccgccgccgccactacaacgactgtttgggctcctaaggcagtgcggtgcacgagaggcttctttttccacgatagagaaactgcaccattggctgctgcggcgcctgctcccgcaggagagctgggagacggagatgacgtggattgcgactactattgttctggttcgagttccgctgcaaccacaacctcctcctcctccctcccggttgtggtcgagccatgctttagtgccggcgatgactggatggatgacgtccgcgccttggcgtcattcctcgacacagatgacgcttggaacctgtgtgcgtga

SEQ ID NO: 12 - DNA, 34 bp, Artificial Sequence
  Synthetic
ggactcttgaccatgatgggacgcagggcttgct

SEQ ID NO: 13 - DNA, 35 bp, Artificial Sequence
  Synthetic
attcgagctggtcactcacgcacacaggttccaag

SEQ ID NO: 14 - DNA, 446 bp, Artificial Sequence
  Synthetic
ttacaagcttcgtgcaggagaggacggtacccacacaatctgccctgttaaccaaattttctgtcagcctcaggggagagggtgaaggtgggccccatacggaaaacttaccatggccttaaatttatttgggcgcgccggaaaactacctgttcacgtgcacttgtcactacttggtgacctgttcaatgcttttcgagctcacccagatcatatgatctagacacgacttcttcaagaggcctcatgcctgagggatacctgcaggagtgtatgagtaaggtactgtcgacggagacaccctcgtccctagggatcgagcttaagggatttaaatatttcaaggaggacggaattccggccacaagttggaactagtactacaactcccacaagcgctactagcagaccattaggatcccattgaagatggaagagatctggca

SEQ ID NO: 15 - DNA, 38 bp, Artificial Sequence
  Synthetic
tgacatgattacgaattaagcttcgtgcaggagaggac

SEQ ID NO: 16 - DNA, 39 bp, Artificial Sequence
  Synthetic
gacggccagtgccaagctagatctcttccatcttcaatg

SEQ ID NO: 17 - DNA, 5866 bp, Artificial Sequence
  Synthetic
ggtcaccgagctctctagacgaaatgctcggttatatgttttccatgtaatcgagaaaacaaatacggagtaataatacagtaattaagagtagcaggaggcagccatagattgcagtcgccctccctatgttccatcacaatatcatgatatgattttcgcggttcatctccaaactccagcgaaagctccgatgctttcggccggtgcgaatcgccggtacatgacatctgatcaacgtgtgcaacgtttacgtttgggcctccaaaatccagccaaaacatggaggttttttttcacactaatacaacggcccatgaacatctccggtcctcagttgcttccataactacaaggcgaaattattattttttttagaaaattctccatgtacctctaaaattttatccaatttcttctatatcagcacttttgaactcattcctttatgtaccccgacttctagtttggattccctctctaaccttttcgtggggtcaaccgttaaattcatattaaaaagtccatttttcctttcagtatagaggaacatatatatatatatatatatatttgtgagtttgttatacgagatattatttatgacaaaatttatttaaaaatatgatatagaaaataatatttatttatctgttattaaaaattgtttacgtaggatataaatagatttgtcttatactacaatcaaaataatacagtacttatataatttttaataactaaaaaaataaatattattctttcatgtcatatataaaaataaattttatcgtaaataatatttttcataacaaatacataaatataaaagtctcgtacaataaatttttatactccaataatatactcataccaaaaggtaaaatggacttttgattaagaatttaatggtcaactaacggaaaagttatgaaagggatctaaactaaaatttagaagtatgcaaggaaataagcaaaacttagtaccacataagagattagacaaagtcttataggtacataagaaatttctcttatttttgggaagcaccagtagtgtcgtttgtggagtcgtaggtgcatgactccacccaccatgctttaaatcctgatgcccacgaatattacgcacatgcatgtgaactttcaataggactttagtgagacgctggtttccgtctctaagagcatgtgttaggagacacatttgttggggtgtgagtatggtgttacgtgtgtagtggtgtgtgtctgcggtataatcttaaaaaaaagttctcttatttttgtggtgcataatttttgacatggaagacctttttctcgtcacagacaagcaccaccgggcaccgactgatttctgaagtcgtcactggttgtccctcgtgaatcgtcgtcacctgctaagcccaaatatccacagaaagttagtggtatcaaattatgttatctccattcgatattttgggtttattttaagtcaaatatattaagtttgatcgagtttgtagaaaaacgtagcaacatctataacatcaaattagttttattgaatccacgattgaatatgttttatagtatatttgttatgtgttaaaaatattgctatatttttttataaattttagtttgacttatgacaaatccaaaatgttttacaatatacgcgtatgacaatctttgaggtgtaggagattaaattctttgcatttgattgtttatacatctagttttgtcacatgaaataacacacagttctgattttcaaagaaaaaaagtaacagataattctgatgtgcatacatggaaggttctcttctgaaagcccttagaaagattgtggttcaccaaacaaggtgcagaaaaaaaacaagaacacaacagtatatatatataacaaagtaggcatgctcttggttggttactactggttctgtttctcatgctcatccatgcatttcttgctacctgattaaaaggagagaagggcagtctgcttggcgctctctctctgaacctgtcactctttttttctgtgtctcttggaggagatgatggagatcagcagcaatgaggagatgatggagatggccattgttgagcagctgcctccttcctctcatcatctcaatggtggcagtgttgaggttgacatggaggaggatcatgtgtggccaaccaaagatggccctctccctatattccttaaggtgtgtatatctatctcttcttccatttttcggtatgcgcgaaaacagatcaagtttatccttgttgatgttcaagaaagtgttttaatccctcgtttatggttatgaaaaattatgaaaacaataaatagattatccctataatcttttataggtaagtggatacatatgtgcatgtttgtaggtatgtgaaaagattaatcagaatgcttacacatgttgatgttctccctatgtagtgtgtgtatatatgtgcatgtttgatgtttgtagcaactgaacaatttagttctactaagcagagtttgaaaaagatcgatattactaccaggatttattaagatactaaaggttttaccgtgattaatcatgaaatcatgaaaaaaagtttgtattaattatttcttatgcattttatagagtctaattttgaactcagagatgcaaaaaatgccaaacaaaggaaacgacctgaaatagacatacttaaggagggaaggtacaagttagttggttggcaatttgcatgtctcattgctcaacagtttcttatctaattgcaaaaacttcttgcatggtgtttttacttctttggcatgcaatatagctaacaaaaaaggagcaaattcctggtctaaatttttgcttgatatataaatatttttttttggaaagtgcagttcgagaacgtcgagtacaaggtgaagttgacaccaaagaacccactgacagccgcccgggtcgcgttcgcgtcacataagtcaaccgaggatcagggctcatgcaagcatattcttaagggcattgggggctccgtggaccctggggagattttggcccttatggggccgtctgggtcaggcaagaccacgctgctgaagatcctcgggggcagactgtctggcggcgttaagggccagatcacctacaacgatacaccgtactctccgtgcctcaagcgcagaatcggcttcgtgacccaggacgatgtgcttttcccacagcttacggtcgaggagaccctcgttttcgccgcgtttcttcgcttgcctgccagaatgtcaaagcagcagaagcgcgatagagtggatgcgattattaccgagctgaaccttgaaaggtgcaggcatacaaagattgggggcgcgttcgttagaggcgtgtctggcggcgagcgcaagcggacgtctatcggctacgagattttggttgatccatcacttcttttgttggatgagccaacatctgggctcgactcaacatcagccgccaagctcctcgtggtgctcagacgcctcgcccggtcagccgcccggagaacagtgatcacaacaatccaccagccatcatctaggatgttccatatgttcgataagctcttgctggttgccgagggccacgcgatttaccacggcggcgccagaggctgcatgagacatttcgccgcccttggcttctcacctggcattgccatgaaccctgcggagttccttttggatctcgcgaccgggaacttggatggcattagctctccagcgtcactgttgctcccgtctgcggccgcggcgtcccctgatagcccggagttccggtcccacgttattaagtacctccaggccagacatcgcgccgccggggaggaggaggccgccgcggccgccgcgagagagggcgggggcgggggcggcgccggcagggacgaggccgccaagcagcttagaatggccgttagaatgcgcaaggatagaagaggcgggattgggtggcttgagcagttcacggtgttgagtagacgcacattcagggagagagccgcggactacctggataagatgagacttgcccagtcagtcggggttgcgctgctgttgggcctcttgtggtggaagtcacagacatctaacgaggcgcagctcagggaccaggtcgggttgattttctacatttgcatcttctggacctcatcgtcgctcttcggctcagtgtacgttttcccattcgagaaactctacctggtgaaggagcgcaaggccgatatgtacagactctctgcgtactacgcgtcttcaacggtgtgcgacgcggtcccacacgtagtttaccctgttttgttcacagcgatcctttacttcatggccgatcttagacgcacggtcccgtgcttctgcctgacacttcttgcgacactccttattgtgctgacatcgcagggcaccggggagttgctcggcgccgcgattctgtccgttaagagagccggggttatggcctcactcgtgcttatgctgttccttttgacaggcggctactacgtccagcatatccctaagttcattaggtggttgaagtacgtgtcattcatgcactacgggttcaaccttctcctgaaggcccagtatcacgggcacttgacatacaactgcgggtctaggggcgggtgccaaaggttgcagtcgtctccatctttcggcacagttgatctcgacggcggcatgagggaggtttggattctcctcgcgatggcggtcgcctacagactccttgcgtacttgtgccttagaaagagaattagcctgatgccactgtgatccatcgatctgtcagatcaggatcagttggttcggggaagaagacgactgatgtggtagtgaattgcaattgcgattgatgatggtgaatgggccattgttggtttgggtgctgtgaattgtttcaaacttgagttcttttctttatttgagaagtatttgaggcttgtctgcttagcaagttgggaaataatcaaccccatcattttagcattcagcttgtaagcataagcgaagatttatttaaaacttatttttaaagttaattttaggtatcttttcgtagtttattttctagcatttactttttaatttgctgagcatgtatattaaagttttacgtataattttttttaaaattattaataagttgtttgtataagcgattagtctgtctcgtcgtcgtgacaaaaagaaaaaccgatgagatggttgggagccgattaaggttattgttttcctcgaaaagagaaggtggaggggatggggtgaatttaactctccctgcaaaaccatttttggctttgaactcaaaatttctcattttccgtttgtaatggacatagtagtatgttctcctcagtgcctttgccctattaggcccaaatctcctgcccgcaaaaaatgggctgacaacgtcacgaaccacgaaccgcgccgtaaatgggccaaaaataccaaggcccaaatcctacagagcccaaagtagagggccataatatgggcctagaaattcaacggcccgctccaatgagaagtggactcggagcgggattcccccaccaaatccgtttcgacctcagaaaatacgcctccgatcaggcgtttacgcacactactaatattgacgagaaagccacctcctctctctcgctcgctccgctcttcttgacaaggaagcaaaagccgccgccgccgacgccgacgagagaggagagagagatcgccgccgacgtcgcaggagggggagggggagatggcggcggagggggaggacgcggcggccgcgcgacgccgcgcggcggccaccgactacaggaagaagctcctcacctgcagggtcgac

SEQ ID NO: 18 - DNA, 3037 bp, Artificial Sequence
  Synthetic
ttacactagtccaccgacttcgaagcaatctgtgcgccggttggaggtggcggagcactggtaccgactctacaagacggacaatcaacgggtaccacaatccggctccacctcgacgcgattgcggcaaccacgaaaacctcacgctccaatctgtgccctccgcccgtgctcgctgcatctcgtgccaccgtctcggactctcgatcctcatggcctgaattatcctaattcttcgttaccagtttttttgagactaatatgactcccatcaaacaatgcagttgagagtgagttcttacctgtattatatagtacattgtatttaaaccatagtacatggggacagtggtgcgttcatcaatttatggattgtggtaggctggtagctgtttgatttgtcactaaaattgcacgacgacaacactttggccctgtttagattctaacttttttcttcaaacttccaacttttccgtcacatcgaacttttctacacacacaaacttccaactttcccgtcaccttcactttaggttcctaagtttaggttaccttcactttaggttcctaaatttatcactaagtctgaaatttatccctaaaccaaaataccaggtacaacggatccctcaatctacaaaactcaatcacccaagattgtagatagtattatgtccggttttaactgacgtgtcaagttgagttaacgtggaatctatgcgggccccacatgtaagtggctagtacttttttccacgttggacgaaaccgtctcccaaaccactaaaggaggcgatatgcaccgattttgatagatgggggagacgttataccctgttttattatcaagagatgtgattcaaccaggagcaagagttgagagagcaagaatagacttattcgtgagcccacaggctaaacccgccacggcccacagactaagcccgccaactcgggccgttcggcccagtttgcacgcatggtgggccggaaactggaatctccgaaccgcaacggaatcgacgacatctgtagccacagcgcgcactcgacggcgggcagcttcgtcactggttcgagctgtctgctgactcaacgacgaacacacgtactcctgctcggtttcttcctccgtgtcgcacaaaagtcaaattgctctctattagtattcattattagcacttcaaacctttctttacgttttaaacgaaccaactaatcaggttatgaggactataataatcgaatccagggatcttgctggaagcaattgaataatcgcccaacgattgagttcatttcttgtctccaaagctgtctcctgatagtcaacaggtctcggtcctcgcagagtcgcactgcgatttggcctctggaaactggaaagagatctcgatccaccacaagaaaatgccaagcagcacgacgacgaagacgacaacgccttccaggtacgtagcaattgaatacggaatactctctccgtttaaaacgtttgattattttcctaatcaaactttatcatgtttgaccaaatttatagaaaaaaataacaacatcttaaatataaaattagtataactaaatctagcattggatatactttcataatatttgtttgttttatgttaaaaatactactatatttttctataagcttagtcaaatttaaatcaaattaaagaagattaattagaaaaatagccaaacgatttgtaatatgcaacggagtgagtagaagtaatcgcccagcctcgccaacgaggcaacgagacccgtaatgcaacgatcgcatctgcgtttcaggcgtcagccatggcgtctgcagagatgcctggattgtctccgcaagatctgatccatttcatctccttctagaagcacaagcgccgctcggtataaaggcagacgcattgtcacaaatagctgcagtgcaccagagtcacagaaacacatcacacattcgtgagctcagcttagccgcagaaggtgatcctgatcaacggcgagttcccgcgcccgcggatcaactgctcgtccaacaacaacatcgtggtgaacgtgttcaacctgctggacgagccgctgctcttcacctggaacgggatgcagcaccgcaagaactcgtggcaggacggcctcgccgggacgcagtgccccatcgcgccgggcaccaactacacgtacaagtggcagcccaaggaccagatcggcagcttcttctacttcccgtcgctggggatgcaccgcgccgccggcggctacggcgggatcagcgtcgtcagccgcctgcacgagctggtgagctagctattacctaatcgatcgatggtcatcgatcatgagatgatgatgatgagatttgtacttaattgtgatctgtatggatgctgttgttgatcaagttcttgcgatcgatcgatctgaattttcaggtttgagggcaggcggctgacgacgctgatcccgccgtagccgccggcggcgcggtgcatccccagcgacgggaagtagaagaagctgccgatctggtccttgggctgccacttgtacgtgtagttggtgcccggcgcgatggggcactgcgtcccggcgaggccgtcctgccacgagttcttgcggtgctgcatcccgttccaggtgaagagcagcggctcgtccagcaggttgaacacgttcaccacgatgttgttgttggacgagcagttgatccgcgggcgcgggaactcgccgttgatcaggatcaccttctgcgctcgaatttccccgatcgttcaaacatttggcaataaagtttcttaagattgaatcctgttgccggtcttgcgatgattatcatataatttctgttgaattacgttaagcatgtaataattaacatgtaatgcatgacgttatttatgagatgggtttttatgattagagtcccgcaattatacatttaatacgcgatagaaaacaaaatatagcgcgcaaactaggataaattatcgcgcgcggtgtcatctatgttactagatcgggggatccggca

SEQ ID NO: 19 - DNA, 311 bp, Artificial Sequence
  Synthetic
gcaggcggctgacgacgctgatcccgccgtagccgccggcggcgcggtgcatccccagcgacgggaagtagaagaagctgccgatctggtccttgggctgccacttgtacgtgtagttggtgcccggcgcgatggggcactgcgtcccggcgaggccgtcctgccacgagttcttgcggtgctgcatcccgttccaggtgaagagcagcggctcgtccagctggttgaacacgttcaccacgatgttgttgttggacgagcagttgatccgcgggcccgggaactcgccgttgatcaggatcaccttctgc

SEQ ID NO: 20 - DNA, 2949 bp, Artificial Sequence
  Synthetic
ttacgtcgacgtcccatgtcaccgacagtactaaatgggtaaagattggataaagtatatggggtatttgtgaggtattattagaaaacttcgtgtggttttgatggacctgttttatgtgttgaaaatatgaatggttatagggtgtgtttgcaagtgcaggatgggaactcatccctcctgcacgcaaaacggagcggctttttaacacatgattaattaaatattagctaatttttttaaaaaaaatggattaatttgatttttttaagcaactttcatatagaaattttttgcaaaaaacacaccgtttaatagtttaaaaacgtgcgcgcgaaaaacgagggagaggggttgggaacatgggtttgcaaacacaaccatagtattggcgattccttttcgtttgagtaaattttacaaaactacaggtattttgaccaaattatcacaaaactacagatttaaggagttgtatcataaaactacacatttagcatcaaatttatcacaaaactgcagattttaggttaagtatcacaaaaatacatatttaatattgaacttatcacaaaactataacttttggagtttaaatccctagcaccattgttatggtggagctataaacattattactttgtgattaaattggttctaaacctttagttttatgataatttagtaactaaacgtgtagttttgtaacacttcatctttaatatgtagttttgtgctaaatttggtgctaaatgtgtaattttgtgatataattccttaaatatgtagttttgtgatagtttggttataatatctgtagttttatgaaatttactcttttcgttttcactgcaatttggaatgatggaattgactagatccggcattaccgatgggctgccgaacgctgtgatgcggttgatcttgagcgatccgggacgccacaagcaccgatgggttctgggagttcatacggctggtgcagcagtgtgtcaatagcagccgggatgtgcgcccaaccatggtcgccgtcgagaggaggatcgaagacatcctgaactcggttgtcaggtcatccaccaccgggttcatgactgccggaggcgacacacccagcaacgagccaaatcgtgaagataacggaaacgagccaaatcccagcaacgagatcgccagggactagtagtacgtacagcagtggtgatttgtcatataggtgtatatcggctgttttcgcatctcaaggcctcaagcagtgtgtgcaatctggagtagtatataaatatgtaaaatgttcatttcgatatactgtcaaatgcgtgtaaattaaccaatgctaaaacaacacactgtgactaaatttactgagttggatgatgaggatgattatgttgcgtgcacacctgatcaggaggacatataatataggccatttgggccgtcttggacaccaccgtttgatttgtatgaagttgggccgaactatgcaagcccagaggcgctgcctctgtgccacggcccacgggcatcgctggatggtcaagcaggtgatcggtggagcgccaatggcggcggcgagacacacagcgcggcgcgcgcgcgaacgtgcggacgcgcgcgccccggccacggccgccgcgctcgtctcctggcctcccgcgcccgctacaaatggcggccccggcgtcccctcctcactccgaagcttcccggttgacgacctctccggtctcccccctcaccccaccgcaacccgggacgtcttccatggccgccgccgccgccgcccccgcctactaaaccaccctacccaccccctccaaactcccacacattacatccttcaaagagagcatcacacacacacacacaccagcctagcgatcacatttccacggcagaaggtgatcctgatcaacggcgagttcccgggcccgcggatcaactgctcgtccaacaacaacatcgtggtgaacgtgttcaaccagctggacgagccgctgctcttcacctggaacgggatgcagcaccgcaagaactcgtggcaggacggcctcgccgggacgcagtgccccatcgcgccgggcaccaactacacgtacaagtggcagcccaaggaccagatcggcagcttcttctacttcccgtcgctggggatgcaccgcgccgccggcggctacggcgggatcagcgtcgtcagccgcctgcacgagctggtgagctagctattacctaatcgatcgatggtcatcgatcatgagatgatgatgatgagatttgtacttaattgtgatctgtatggatgctgttgttgatcaagttcttgcgatcgatcgatctgaattttcaggtttgagggcaggcggctgacgacgctgatcccgccgtagccgccggcggcgcggtgcatccccagcgacgggaagtagaagaagctgccgatctggtccttgggctgccacttgtacgtgtagttggtgcccggcgcgatggggcactgcgtcccggcgaggccgtcctgccacgagttcttgcggtgctgcatcccgttccaggtgaagagcagcggctcgtccagctggttgaacacgttcaccacgatgttgttgttggacgagcagttgatccgcgggcccgggaactcgccgttgatcaggatcaccttctgcgctcgaatttccccgatcgttcaaacatttggcaataaagtttcttaagattgaatcctgttgccggtcttgcgatgattatcatataatttctgttgaattacgttaagcatgtaataattaacatgtaatgcatgacgttatttatgagatgggtttttatgattagagtcccgcaattatacatttaatacgcgatagaaaacaaaatatagcgcgcaaactaggataaattatcgcgcgcggtgtcatctatgttactagatcggggaattcggca